(19) United States
(12) Patent Application Publication
(10) Pub. No.: US 2011/0294791 A1
(43) Pub. Date: Dec. 1, 2011

(54) PIRENZEPINE AS AN AGENT IN CANCER TREATMENT

(75) Inventor: Andre Schrattenholz, Mainz (DE)

(73) Assignee: PROTEOSYS AG, Mainz (DE)

(21) Appl. No.: 13/144,349

(22) PCT Filed: Jan. 13, 2010
(86) PCT No.: PCT/EP2010/050350

§ 371 Date: Jul. 13, 2011

(30) Foreign Application Priority Data
Jan. 13, 2009 (US) 61144204
Apr. 6, 2009 (US) 61166839

(51) Int. Cl. A61K 31/551; A61P 35/00; C07D 471/04

(52) U.S. Cl. 514/220; 540/495

(57) ABSTRACT

The present invention generally relates to the neuroprotective activity of condensed diazepinones, e.g. condensed benzodiazepinones such as pirenzepine or compounds which are metabolized to condensed benzodiazepinones such as olanzapine. These compounds are suitable as co-medicaments for the prevention and/or treatment of drug-induced neurotoxic effects in general and neurotoxic side effects during cancer treatments with cytostatic drugs such as platinum-derivatives, e.g. cis-, carbo- and oxaliplatin, taxanes, bleomycin, cyclophosphamide and vincristine etc. Further, these compounds have an intrinsic anti-cancer activity on their own due to PARP-1 inhibition, which prevents NADH depletion in oxidative metabolism of healthy cells thus preventing the shift to anoxygenic, glycolytic metabolism present in many types of tumour cells thus eliminating this crucial metabolic advantage favoring tumour growth. These results exploit the fact of differential PARP-1 expression between many cancer cells and healthy tissues.

The present invention generally relates to the neuroprotective activity of condensed diazepinones, e.g. condensed benzodiazepinones such as pirenzepine or compounds which are metabolized to condensed benzodiazepinones such as olanzapine. These compounds are suitable as co-medicaments for the prevention and/or treatment of drug-induced neurotoxic effects in general and neurotoxic side effects during cancer treatments with cytostatic drugs such as platinum-derivatives, e.g. cis-, carbo- and oxaliplatin, taxanes, bleomycin, cyclophosphamide and vincristine etc. Further, these compounds have an intrinsic anti-cancer activity on their own due to PARP-1 inhibition, which prevents NADH depletion in oxidative metabolism of healthy cells thus preventing the shift to anoxygenic, glycolytic metabolism present in many types of tumour cells thus eliminating this crucial metabolic advantage favoring tumour growth. These results exploit the fact of differential PARP-1 expression between many cancer cells and healthy tissues.

Pirenzepine(5,11-dihydro-11[(4-methyl-1-piperazinyl)-acetylJ-6H-pyrido-[2,3-b]-[1,4] benzodiazepine-6-one), is a topical antiulcerative M1 muscarinic antagonist, that inhibits gastric secretion at lower doses than are required to affect gastrointestinal motility, salivary, central nervous system, cardiovascular, ocular, and urinary function. It promotes the healing of duodenal ulcers and due to its cytoprotective action is beneficial in the prevention of duodenal ulcer recurrence. It also potentiates the effect of other antiulcer agents such as cimetidine and ranitidine. It is generally well tolerated by patients. The M1 muscarinic effect of pirenzepine is thought to be an explanation for this and a variety of additional effects in other indications, listed below.

WO 2006/008118 and WO 2006/008119 describe that pirenzepine and related compounds are inhibitors of PARP and SIR2. The use of these compounds as cytoprotective, particularly neuroprotective agents, is disclosed. The contents of these documents is herein incorporated by reference.

The administration of ototoxic or neurotoxic agents during cancer treatments may mediate apoptosis and/or necrosis of sensoric cells due to oxidative stress (Henderson et al., Ear Hear. 27 (2006), 1-19). In early stages of apoptosis a massive activation of PARP-1 was detected (Yu et al., Science 297 (2002), 259-263). Further, it was found that PARP-1 activation causes a translocation of AIF (Apoptosis Inducing Factor) from the mitochondriae to the nucleus and an AIF-mediated PARP-1 dependent caspase-independent apoptosis (Yu et al., (2002), supra). PARP-1 hyperactivity is also associated with necrotic cell death (Virag and Szabo, Pharmacol Rev. 54 (2002), 375-429). Further it could be shown that the PARP-1 inhibitor 3-aminobenzamide alleviates cochleal dysfunctions induced by transient ischemia or acoustic trauma (Tabuchi et al., Ann. Otol. Rhinol. Laryngol. 110 (2001), 118-121; Tabuchi et al., J. Exp. Med. 200 (2003), 1995-2002).

Thus PARP-1 inhibition is a general neuroprotective principle, attenuating the intrinsic, i.e. mitochondrial pathway of apoptosis, which can be induced by a variety of pathological or toxic conditions (Schrattenholz A and \check{S}o\check{s}kicć V, 2006, Current Topics in Medicinal Chemistry, 6(7), 663-686).

Higher expression of PARP in cancer compared with normal cells has been linked to drug resistance and the overall ability of cancer cells to survive genotoxic stress (Virag L, Szabo C. The therapeutic potential of poly(ADP-ribose) polymerase inhibitors. Pharmacol Rev 2002; 54:375-429; Tulin A, Chinenov Y, Spradling A. Regulation of chromatin structure and gene activity by poly(ADP-ribose) polymerases. Curr Top Dev Biol 2003; 56:55-83. 7. Tomoda T, Kurashige T, Moriki T, et al. AmJHematol 1991; 37:223). Enhanced PARP-1 expression and/or activity has been shown in several hematologic and solid tumors. This differential expression of PARP in normal versus tumor cells supports the observed selectivity of PARP inhibitors to affect proliferating tumor cells (Donawho et al., Clin Cancer Res 2007; 13(9), 2728). Therefore, inhibition of PARP sensitizes tumor cells to cytotoxic agents that induce DNA damage that would normally be repaired through the base excision repair system (e.g., DNA glycosylase, AP endonuclease, XRCC1, etc.). The most notable agents in this group are alkylators (e.g., temozolomide and cyclophosphamide), topoisomerase I poisons (irinotecan and camptothecin), and certain types of intercalators (e.g., bleomycin). As a result of chemopotentiation and radiopotentiation as well as potential for single-agent activity of PARP inhibitors, several PARP inhibitors have entered clinical trials for the treatment of cancer (Sheridan C. Genentech raises stakes on PARP inhibitors. Nat Biotechnol 2006; 24: 1179-80; Plummer E R. Inhibition of polyADP-ribose polymerase in cancer. Curr Opin Pharmacol 2006; 6:364-8).

According to the present invention it was found that Pirenzepine and related compounds show significant potentiating anti-cancer activity upon co-administration with cytotoxic drugs such as cisplatin in an animal model of human non-small cell lung carcinoma, and also show a significant anti-cancer activity by itself.

Thus, a first aspect of the present invention relates to the use of a compound of formula I

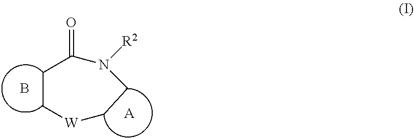

wherein A and B are five- or six-membered rings optionally containing at least one heteroatom selected from N, S and O, wherein the rings are optionally mono- or polysubstituted with halo, e.g. F, Cl, Br, or I, C₁-C₄-(halo)-alkyl, C₁-C₄-(halo)-alkoxy, amino, C₁-C₄-alkyl-amino, or di(C₁-C₄-alkyl)amino,

W is S, O, NR¹ or CHR¹

R1 is hydrogen, Y or COY,

R2 is hydrogen or C₁-C₄-(halo)-alkyl, and

Y is C₁-C₆(halo)alkyl, or C₃-C₈cyclo-(halo)-alkyl, wherein the alkyl or cycloalkyl group is optionally substituted with a five- or six-membered ring optionally containing at least one heteroatom selected from N, S and O, and wherein the ring is optionally mono- or poly-substituted with halo, C₁-C₄-(halo)alkyl, C₁-C₄(halo)alkoxy, amino, C₁-C₄-alkyl amino, di(C₁-C₄-alkyl)amino or Z,

wherein Z is a C₁-C₆(halo) alkyl group ω-substituted with a group N(R4)₂, wherein each R4 is independently hydrogen, C₁-C₈ alkyl, or CO—C₁-C₈-alkyl or wherein both R4 together form a five- or six-membered ring optionally containing at least one further heteroatom selected from N, S and O,

wherein the ring is optionally mono- or polysubstituted with halo, C₁-C₄(halo)-alkyl and C₁-C₄(halo) alkoxy,

or of a salt or derivative thereof for the manufacture of an cytostatic medicament, particularly for the prevention and/or treatment of cancer.

A further aspect of the present invention refers to the use of a compound of formula I or of a salt or derivative thereof for the manufacture of a neuroprotective medicament.

The compound may be used in combination with other medicaments, e.g. anti-cancer medicaments, and/or in combination with radiotherapy. Alternatively, the compound may be used alone without further medication.

The term “(halo)alkyl” according to the present invention relates to an alkyl group which optionally contains at least one halo, e.g. F, Cl, Br or I substituent up to perhalogenation.

The term “salt” preferably refers to pharmaceutically acceptable salts of compounds of Formula I with suitable cations and/or anions. Examples of suitable cations are alkaline metal cations such as Li⁺; Na⁺ and K⁺, alkaline earth metal cations such as Mg⁺ and Ca⁺ as well as suitable organic cations, e.g. ammoniums or substituted ammonium cations. Examples of pharmaceutically acceptable anions are inorganic anions such as chloride, sulfate, hydrogen sulfate, phosphate or organic cations such as acetate, citrate, tartrate, etc.

Derivatives of compounds of Formula I are any molecules which are converted under physiological conditions to a compound of Formula I, e.g. esters, amides etc. of compounds of Formula I or molecules which are products of metabolization reactions of a compound of Formula I.

Preferably, the compounds of Formula I are used for the prevention or treatment of PARP-1 associated proliferative disorders, i.e. all types of cancers which are caused by and/or accompanied by enhanced PARP-1 expression and/or activity and/or apoptosis, in particular mitochondrial apoptosis and/or anoxygenic/glycolytic types of cancer-related metabolism. For example, these disorders are selected from solid cancers such as cancers of brain, breast, colon, stomach, lung, pancreas, prostate, cervix, ovary, esophagus, skin, kidney, liver etc., or cancers of lymphocytes such as lymphomas, or myelomas etc.

In a further preferred embodiment, the compounds of formula I provide protection against neurotoxicity caused by administration of cytotoxic compounds, e.g. administration of chemotherapeutic agents, particularly platinum compounds such as cis-, carbo- and oxaliplatin, taxanes, topoisomerase 1 inhibitors, intercalators like bleomycin, cyclophosphamide and vincristine etc. in cancer therapy.

Further, the compounds of Formula I have a chemopotentiating activity, e.g. increasing the efficacy of chemotherapeutic agents and/or enabling lower dosages of chemotherapeutic agents, particularly for the treatment of cancer as indicated above. For example, the dosage of chemotherapeutic agents may be reduced at least 20%, at least 30%, at least 40% or at least 50% and e.g. up to 60%, up to 75% or up to 90%.

Furthermore, the compounds of Formula I have radiopotentiating activity, e.g. enhancing effects of radiation therapy of cancer and/or enabling lower dosages of radiation, particularly for the treatment of cancer as indicated above. For example, the dosage of radiation may be reduced at least 20%, at least 30%, at least 40% or at least 50% and e.g. up to 60%, up to 75% or up to 90%.

Furthermore, the compounds of Formula I have anti-cancer activity on their own and thus may be administered without further medication, particularly for the treatment of cancer as indicated above.

It was found that compounds of formula I prevent an irreversible loss of neuronal cells, which may be caused by and/or accompanied by administration of chemotherapeutic compounds or radiation therapy.

Particularly, the compounds of formula I may be administered to a subject who is under treatment with medicaments having neurotoxic side effects, e.g. platinum compounds or other chemotherapeutic agents and/or under treatment with radiotherapy, in order to reduce and/or abolish the neurotoxic side effects of such compounds.

Surprisingly, it was found that administration of the compounds of formula I does not negatively affect the cytotoxic anti-tumor activity of chemotherapeutic agents, e.g. cis-platinum, or radiation therapy. In contrast thereto, the compounds of formula I synergistically increase the activity of chemotherapeutic agents and have an anti-cancer effect on their own.

In the compounds of Formula I, the cyclic groups A and B are preferably selected from

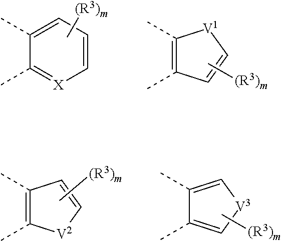

wherein X is N or CR3,

V1, V2 or V3 are selected from —O—, —S—, and NR6,

R3 is in each case independently halo, C₁-C₄-(halo)-alkyl, C₁-C₄-(halo)-alkyl, C₁-C₄-(halo)alkoxy, amino, C₁-C₄-alkyl-amino, or di(C₁-C₄alkyl)amino,

m is an integer of 0-2, and

R6 is hydrogen or C₁-C₄-(halo)alkyl.

More preferably, the cyclic group A is selected from

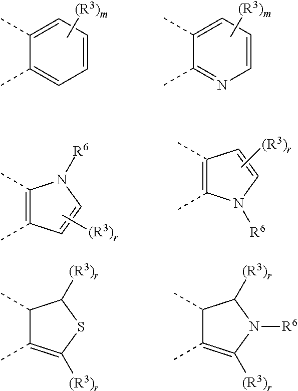

wherein R3 is defined as above,

m is an integer of 0-2,

r is an integer of 0-1 and

R6 is hydrogen or methyl.

More preferably, the cyclic group B is selected from

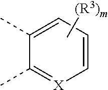

wherein X, R3 and m are as defined above

In one embodiment, R1 is Y. In this case Y is preferably C₃-C₈ cyclo(halo)-alkyl, e.g. cyclopropyl, cyclobutyl or cyclopentyl.

In a further embodiment, R1 is COY and Y is selected from

—(CHR7)q-R8

wherein R7 is hydrogen, halo or C₁-C₄-(halo)alkyl,

q is an integer of 1-4, and preferably 1 and

R8 is a five- or six-membered ring optionally containing at least one heteroatom, wherein the ring is optionally mono-or polysubstituted with C₁-C₄(halo)alkyl or a w-amino-substituted alkyl group Z as defined above.

In this embodiment, R8 is preferably selected from

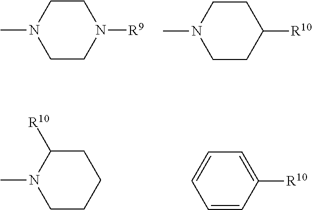

wherein R9 is hydrogen or C₁-C₄(halo)alkyl and R10 is a w-amino-substituted

alkyl group Z as defined above.

R9 is preferably a methyl group. The w-amino-substituted alkyl group Z is preferably a C₁-C₄(halo)alkyl group having a terminal amino group which is substituted with at least one C₁-C₆ alkyl group, e.g. a diethylamino, or di-isobutylamino group, or with a CO(C₁-C₆) alkyl group and with hydrogen or a C₁-C₂ alkyl group.

Specific examples of compounds of Formula I are pirenzepine and related compounds as disclosed in FR 1,505,795, U.S. Pat. Nos. 3,406,168, 3,660,380, 4,021,557, 4,210,648, 4,213,984, 4,213,985, 4,277,399, 4,308,206, 4,317,823, 4,335,250, 4,424,222, 4,424,226, 4,724,236, 4,863,920, 5,324,832, 5,620,978, 6,316,423, otenzepad and related compounds as disclosed in U.S. Pat. No. 3,406,168, 5,324,832 and 5,712,269, AQ-RA741 and related compounds as disclosed in U.S. Pat. Nos. 5,716,952, 5,576,436 and 5,324,832, viramune and related compounds as disclosed in EP-A-0429987, and U.S. Pat. Nos. 5,366,972, 5,705,499, BIBN 99 and related compounds as disclosed in U.S. Pat. Nos. 6,022,683 and 5,935,781, DIBD, telenzepine and related compounds as disclosed in EP-A-0035519, and U.S. Pat. No. 4,381,301 and salts or derivatives thereof. The above documents are herein incorporated by reference.

Further preferred compounds are 7-azabicyclo-[2.2.1]-heptane and heptene compounds such as a tiotropium bromide as disclosed in U.S. Pat. Nos. 5,817,679, 6,060,473, 6,077,846, 6,117,889, 6,255,490, 6,403,584, 6,410,583, 6,537,524, 6,579,889, 6,608,055, 6,627,644, 6,635,658, 6,693,202, 6,699,866 and 6,756,392, heterocyclic compounds, e.g. pyrrolidinones, tetrahydropyridines, isoxazocarboxamides, thienopyrane carboxamides, or benzopyranes, such as alvameline tartrate and related compounds disclosed in U.S. Pat. Nos. 6,306,861, 6,365,592, 6,403,594, 6,486,163, 6,528,529, 6,680,319, 6,716,857 and 6,759,419, metoclopramide and related compounds as disclosed in U.S. Pat. No. 3,177,252 and QNB and related compounds as disclosed in U.S. Pat. No. 2,648,667 and salts and derivatives thereof. The above documents are herein incorporated by reference.

Further, the invention encompasses compounds which are metabolized to give diaryl diazepinones according to Formula I such as clozepine and olanzapine.

The compounds as indicated above are preferably administered to a subject in need thereof, e.g. a human subject, as a pharmaceutical composition, which may contain pharmaceutically acceptable carriers, diluents and/or adjuvants. The pharmaceutical composition may be administered in the form of a tablet, capsule, solution suspension, etc. The medicament may be administered according to any known means, wherein oral and intravenous administration is particularly preferred. Alternatively, the medicament may be administered via nasal sprays or depots.

The present application has applications in human and veterinary medicine, particularly in human medicine.

Furthermore, the present invention shall be explained by the following Figures and Examples.

### FIGURE LEGENDS

FIG. 1 shows Anti-Tumor activity of pirenzepine (PSY 310) in the H 460 xenograft model of human lung cancer.

### EXAMPLES

### Example 1

### Cytostatic Activity of PSY 310 (Pirenzepine) in Reducing Tumour Growth

It was investigated whether compound PSY 310 has effects on in vivo efficacy of cisplatin in the lung tumor xenograft model H460. PSY 310 was tested with 2 different dosing schedules of cisplatin.

1. Experimental Protocol

Study Design Tumor Inoculation:
Animals: Female NMRI nude mice (Janvier, Le Genest St Isle, France), age: 6-7 weeks at start of experiment Janvier, Le Genest St Isle, France; age 5-6 weeks). 70 animals were inoculated at the beginning of the experiment.
Cells: Human H460 Large Cell Lung Cancer (ATCC: HTB-177);
   3×10⁶ cells from cell culture was inoculated subcutaneously per mouse in a volume of 200 μl on day 0.
Application route: subcutanously;
   1 tumor/mouse
Tumor volume at
start of therapy: 62.5-100 mm³
   (formula: a×b²×0.5; a: length, b width)

Study Design Therapy:
Test compounds: Cisplatin,
   PSY 310 (Pirenzepine)
Dosing: Cisplatin
   1) high dose: 1×/week, 2 weeks (days 7, 14)
   dose: 2.5 mg/kg/day; route: ip
   2) low dose 5×/week, 2 weeks (days 7-11+14-18)
   dose: 0.15 mg/kg/day; route: ip
   PSY 310
   1×/day for 12 days (days 7-19)
   dose: 50 and 20 mg/kg/day; route: po
Vehicle/solvent: Cisplatin: water for injection
   PSY 310: PBS, oral gavage
Application volume: 10 ml/kg
Criteria for study termination: Animal weight loss >20%
   Tumor>10% body weight or ulceration of tumors
Parameters: Survival, body weight, tumor growth (Table I)
Number of groups: 7
Animals per group: 10
   (8-10 mice with subcutaneous tumors of 62.5-100 mm³ will be selected) Table II
Total animal number: 70

TABLE I
Parameters

| | |
|---|---|
| Survival | Daily |
| Clinical signs | Daily |
| Necropsy | dead animals and at the end of |
| | experiment |
| Tumor volume | Start of treatment (tumor size: 62- |
| Start experiment at: approx. 7-10 | 100 mm³) on day 7 |
| days after tumor inoculation | 2x/week |
| During experiment individually | |
| monitored and recorded | |
| Body weight | Start of treatment, then 2x/week |

TABLE II
Experimental groups

| | | | Dose | Animals |
|---|---|---|---|---|
| Group | Compound | Dosing | (mg/kg/day) | (n) |
| 1 | Control (0.9% | 1x/week ip, 2 | 0 | 8-10 |
| | NaCl) + | weeks | | |
| | Vehicle PSY 310 | 1x/day, 12 days po | | |
| 2 | Cisplatin (Cpt) | 1x/week ip, 2 | 2.5 | 8-10 |
| | | weeks | | |
| 3 | Cisplatin (Cpt) | 5x/week ip, 2 | 0.15 | 8-10 |
| | | weeks | | |
| 4 | PSY 310 | 1x/day, 12 days po | 50 | 8-10 |
| 5 | Cpt + | 1x/week ip, 2 | 2.5 | 8-10 |
| | PSY 310 | weeks | | |
| | | 1x/day, 12 days po | 20 | |
| 6 | Cpt + | 1x/week ip, 2 | 2.5 | 8-10 |
| | PSY 310 | weeks | | |
| | | 1x/day, 12 days po | 50 | |
| 7 | Cpt + | 5x/week ip, 2 | 0.15 | 8-10 |
| | PSY 310 | weeks | | |
| | | 1x/day, 12 days po | 50 | |

2. Methods

NCI-H460 human NSCLC cells were implanted in the flanks of immunodeficient mice and growth of the resultant solid tumors was recorded. Mice were assigned to seven treatment groups as indicated above.

Tumor dimensions (and animal body weights) were measured on days 10, 14, 17, and 21 after inoculation, and tumor volumes were calculated.

As tumor growth was close to exponential evaluations were performed on the common logarithms of tumor volumes in order to render the data suitable for evaluation by a linear model. Initial tumor volumes exhibited an enormous variation among animals. In order to render tumor growth comparable between animals, the log(initial volume) was subtracted from each log(volume). Alternatively, the ratios log(volume)/log(initial volume) were employed, yielding very similar results. Tumors with extremely small initial volumes (<20 μl) in which volume measurements are expected to be rather imprecise were omitted from the evaluations.

Comparisons were carried out in the following way:
The slopes of log(volume) over time (i.e. the exponent of the approximately exponential volume growth) were determined for each animal. The ratios of the exponents averaged over the respective groups are given as a measure of the magnitude of the growth retarding effect of the current treatment.
Multivariate variance analyses using the day of measurement and Cisplatin/PSY 310 treatment as model effects were performed.

3. Results

The following results on differences in tumor growth rates were obtained:

| 1. Cisplatin_low + PSY_310_50 | < Cisplatin_low | <> Control |
|---|---|---|
| exponent ratio | 0.65 | 0.85 |
| ANOVA | p = 0.0048 | p = 0.077 |

| exponent ratio | 0.55 | |
|---|---|---|
| ANOVA | p < 0.0001 | |
| 2. Cisplatin_high + PSY_310_50 | < Cisplatin_high | < Control |
| exponent ratio | 0.91 | 0.81 |
| ANOVA | p = 0.0066 | p = 0.014 |

| | |
|---|---|
| exponent ratio | 0.74 |
| ANOVA | p < 0.0001 |

| 3. PSY_310_50 | < Cisplatin_high | <> Cisplatin_low | <> Control |
|---|---|---|---|
| exponent ratio | 0.74 | 0.96 | 0.85 |
| ANOVA | p = 0.016 | p = 0.91 | p = 0.077 |

| | |
|---|---|
| exponent ratio | 0.61 |
| ANOVA | p = 0.0003 |

In consequence, treatment with 50 mg/kg/d PSY 310 alone is not only effective (p=0.0003) but is even more effective than cisplatin-low dose and cisplatin-high dose regimens. Combined treatment with cisplatin and PSY 310 gives the best results.

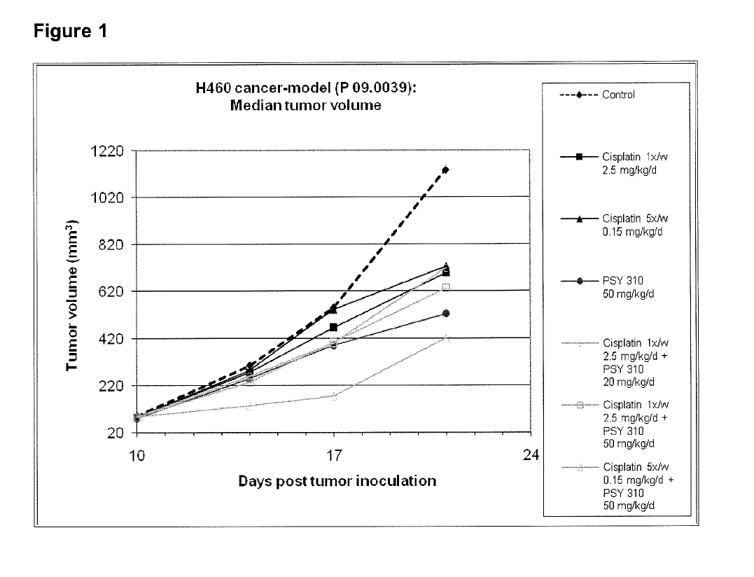

The results are summarized in FIG. 1. It can be gathered that PSY 310 is potentiating the cytostatic effects of cisplatin and has single agent cytostatic properties by itself.

Taken together, these results suggest that cisplatin has moderate effects upon tumor growth in this model, while a major effect is brought about by PSY 310 (50 mg/kg/d) or by co-administration of PSY 310 and cisplatin.

1. Use of a compound of formula I 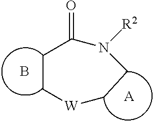
wherein A and B are a five- or six-membered ring optionally containing at least one heteroatom selected from N, S and O, wherein the ring is optionally mono- or polysubstituted with halo, C₁-C₄-(halo)-alkyl, C₁-C₄-(halo)alkoxy, amino, C₁-C₄-alkyl-amino, or di(C₁-C₄-alkyl)amino,
W is S, O, NR₁ or CHR₁
R1 is hydrogen, Y or COY,
R2 is hydrogen or C₁-C₄-(halo)-alkyl, and
Y is C₁-C₆(halo)alkyl, or C₃-C₈cyclo-(halo)-alkyl, wherein the alkyl or cycloalkyl group is optionally substituted with a five- or six-membered ring optionally containing at least one heteroatom selected from N, S and O, wherein the ring is optionally mono- or poly-substituted with halo, C₁-C₄-(halo)alkyl, C₁-C₄(halo)alkoxy, amino, C₁-C₄-alkyl amino, di(C₁-C₄-alkyl)amino or Z,
wherein Z is a C₁-C₆(halo)alkyl group an ω-substituted with a group N(R4)₂,
wherein each R4 is independently hydrogen, C₁-C₈ alkyl, or CO—C₁-C₈-alkyl or wherein both R4 together form a five- or six-membered ring optionally containing at least one further heteroatom selected from N, S and O, wherein the ring is optionally mono- or polysubstituted with halo, C₁-C₄(halo)-alkyl and C₁-C₄(halo) alkoxy,
or of a salt or derivative thereof for the manufacture of a cytostatic medicament.

2. The use of claim 1 for the manufacture of a medicament for the prevention or treatment of cancer.

3. The use of claim 1 for the co-administration with a further medicament and/or with radiation treatment.

4. The use of claim 3 wherein the further medicament is a chemotherapeutic agent, particularly a platinum compound such as cisplatin, carboplatin or oxaliplatin.

5. The use of claim 1 wherein the compound of formula I has chemopotentiating and/or radiopotentiating activity.

6. The use of claim 1 for administration as a single medicament.

7. Use of a compound of formula I as defined in claim 1 or of a salt or derivative thereof for the manufacture of a neuroprotective medicament.

8. The use of claim 7 for the manufacture of a medicament for the prevention or treatment of neurotoxicity caused by administration of chemotherapeutic agents, particularly platinum compounds such as cis-platin, carboplatin, or oxaliplatin.

9. The use of claim 1 for administration to a subject who is under treatment of medicaments having neurotoxic side effects.

10. The use of claim 1 wherein the cyclic groups A and B are selected from 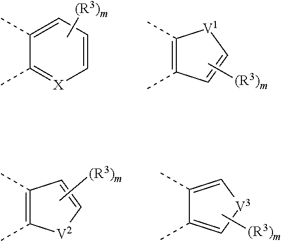
wherein X is N or CR3,
V1, V2 or V3 are selected from —O—, —S—, and NR6,
R3 is halo, C₁-C₄-(halo)-alkyl, C₁-C₄-(halo)-alkoxy, amino, C₁-C₄-alkyl-amino, or di(C₁-C₄-alkyl)amino,
m is an integer of 0-2, and
R6 is hydrogen or C₁-C₄-(halo)alkyl.

11. The use of claim 9, wherein the cyclic groups A and B are selected from 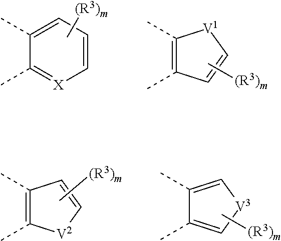
wherein R3 is halo, C₁-C₄-(halo)-alkyl, C₁-C₄-halo-alkoxy, amino, C₁-C₄-alkyl-amino, or di(C₁-C₄-alkyl)amino,
m is an integer of 0-2,
r is an integer of 0-1 and
R6 is hydrogen or methyl.

12. The use of claim 1 wherein R1 is Y and Y is C₃-C₈-cyclo(halo)alkyl.

13. The use of claim 1 wherein R1 is COY and Y is selected from
—(CHR7)q-R8
wherein R7 is hydrogen, halo or C₁-C₄-(halo)alkyl,
q is an integer of 1-4, and preferably 1 and
R8 is a five- or six-membered ring optionally containing at least one heteroatom,
wherein the ring is optionally mono-or polysubstituted with C₁-C₄(halo)alkyl or an ω-amino-substituted alkyl group Z as defined in claim 1.

14. The use of claim 13 wherein R8 is selected from 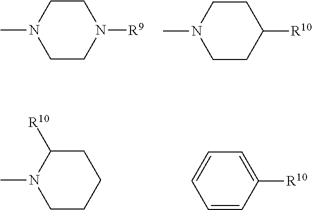
wherein R9 is hydrogen or C₁-C₄(halo)alkyl and R10 is an ω-amino-substituted alkyl group Z, wherein Z is a C₁-C₆(halo)alkyl group an ω-substituted substituted with a group N(R4)₂, wherein each R4 is independently hydrogen, C₁-C₈ alkyl, or CO—C₁-C₈-alkyl or wherein both R4 to ether form a five- or six-membered ring optionally containing at least one further heteroatom selected from N, S and O, wherein the ring is optionally mono- or polysubstituted with halo, C₁-C₄(halo)-alkyl and C₁-C₄(halo)-alkoxy.

15. The use of claim 1 wherein the compound of Formula I is selected from pirenzepine LS-75, otenzepad, AQ-RA741, viramune, BIBN 99, DIBD, telenzepine and salts or derivatives thereof.

16. The use of claim 1 for use in human medicine.

17. A method of treating cancer in a patient in need of such treatment, comprising administering to said patient an effective amount of at least one compound of formula 1 of claim 1.
